(19) United States
(12) Patent Application Publication
(10) Pub. No.: US 2021/0248684 A1
(43) Pub. Date: Aug. 12, 2021

(54) METHODS FOR MORE ACCURATELY MANAGING PROCESSING OF MEDICAL BILL DATA AND DEVICES THEREOF

(71) Applicant: Mitchell International, Inc., San Diego, CA (US)

(72) Inventors: Michele Hibbert Iacobacci, San Diego, CA (US); Susan Englehart, San Diego, CA (US); Edward Olsen, San Diego, CA (US)

(73) Assignee: Mitchell International, Inc., San Diego, CA (US)

(21) Appl. No.: 17/243,412

(22) Filed: Apr. 28, 2021

Related U.S. Application Data
(63) Continuation-in-part of application No. 16/163,165, filed on Oct. 17, 2018.
(60) Provisional application No. 62/573,246, filed on Oct. 17, 2017.

(51) Int. Cl. G06Q 40/08; G06Q 10/10

(52) U.S. Cl. CPC G06Q 40/08; G06Q 10/10

(57) ABSTRACT

Methods, non-transitory computer readable media, and computing apparatus that assist with more accurately managing processing of medical bill data includes identifying previously submitted bill data associated with received medical bill data from a client based on one or more service data parameters in the received medical bill data and the identified previously submitted bill data. The received medical bill data is determined to be erroneous medical bill data based on the identified previously submitted bill data and a time period between the previously submitted bill data and the received medical bill data. The received medical bill data is classified as a follow-up procedure bill data when the received medical bill data is determined to be an erroneous medical bill data. Compensation data is restricted for the received medical bill data classified as the follow-up procedure bill data.

### CROSS-REFERENCE TO RELATED APPLICATIONS

This application is a continuation-in-part of U.S. patent application Ser. No. 16/163,165, filed on Oct. 17, 2018, which claims the benefit of U.S. Provisional Patent Application Ser. No. 62/573,246, filed Oct. 17, 2017, which are hereby incorporated by reference in their entirety for all purposes.

### FIELD

This technology relates to methods and devices for more accurately managing processing of medical bill data and devices thereof.

### BACKGROUND

When a surgical procedure is completed, the healthcare provider is authorized to submit billing data for processing and reimbursement. The date of the surgical procedure starts a clock for the healthcare provider to submit any procedures related to the surgical procedure(s), known as the global surgical period. The date of the surgical procedure however, any follow-up procedures that occur during the global surgical period (varies depending on severity of the procedure, typically 60 days) should not be included with the billing data being submitted for the completed surgical procedure and must be processed separately. By way of example, follow-up procedures after a surgical procedure may include dressing changes or office visits. Procedures unrelated to the surgical procedure can be billed and paid for separately and must be documented by the provider.

Unfortunately, existing technologies to process medical billing data are currently unable to effectively identify follow-up procedure billing data for these follow-up procedures from surgical procedure billing data. Additionally, healthcare providers are sometime delinquent in making a timely submission of billing data for the surgical procedure, e.g. sometimes after the global surgical period has started, and as a result billing data for follow-up procedures is incorrectly identified and processed under standards for normal office visits for payment. Accordingly, previously existing technologies have failed to provide any technical solutions for addressing these issues with billing data resulting in numerous and significant billing payment errors.

### SUMMARY

A method for more accurately managing processing of medical bill data includes identifying previously submitted bill data associated with received medical bill data from a client based on one or more service data parameters in the received medical bill data and the identified previously submitted bill data. The received medical bill data is determined to be erroneous medical bill data based on the identified previously submitted bill data and a time period between the previously submitted bill data and the received medical bill data. The received medical bill data is classified as a follow-up procedure bill data when the received medical bill data is determined to be an erroneous medical bill data. Compensation data is restricted for the received medical bill data classified as the follow-up procedure bill data.

A non-transitory computer readable medium having stored thereon instructions for more accurately managing processing of medical bill data comprising machine executable code which when executed by at least one processor, causes the processor to identify previously submitted bill data associated with received medical bill data from a client based on one or more service data parameters in the received medical bill data and the identified previously submitted bill data. The received medical bill data is determined to be erroneous medical bill data based on the identified previously submitted bill data and a time period between the previously submitted bill data and the received medical bill data. The received medical bill data is classified as a follow-up procedure bill data when the received medical bill data is determined to be an erroneous medical bill data. Compensation data is restricted for the received medical bill data classified as the follow-up procedure bill data.

An insurance data management computing apparatus including at least one of configurable hardware logic configured to be capable of implementing or a processor coupled to a memory and configured to execute programmed instructions stored in the memory to identify previously submitted bill data associated with received medical bill data from a client based on one or more service data parameters in the received medical bill data and the identified previously submitted bill data. The received medical bill data is determined to be erroneous medical bill data based on the identified previously submitted bill data and a time period between the previously submitted bill data and the received medical bill data. The received medical bill data is classified as a follow-up procedure bill data when the received medical bill data is determined to be an erroneous medical bill data. Compensation data is restricted for the received medical bill data classified as the follow-up procedure bill data.

This technology provides a number of advantages including providing methods, non-transitory computer readable media, and apparatus that effectively assists with more accurately managing processing of medical bill data. By using the disclosed technique, erroneous bills, such as fraudulent or incorrect billing data, can be concurrently assessed rather than after the fact to prevent improper payments. Further, the disclosed technology assists insurances companies from paying claims which either are not owed or are processed incorrectly under the wrong standard.

### BRIEF DESCRIPTION OF THE DRAWINGS

FIG. 1 is an example of a block diagram of an insurance data management computing apparatus for identifying and preventing fraudulent medical data;

FIG. 2 is an example of a block diagram of an insurance data management computing apparatus;

FIG. 3 is an exemplary flowchart of a method for identifying and preventing fraudulent medical data;

FIG. 4A is an exemplary post-surgical rule; and

FIG. 4B is an exemplary adjustment made to a medical bill.

### DETAILED DESCRIPTION

Once healthcare providers submit billing data for processing and reimbursement after the surgical procedure, there may be time urgency rules for processing and reimbursing the bill under regulatory or auto policy contracts. For example, the computer system may be limited to a reimbursement period (e.g., 10-30 days) to pay the bill. The reimbursement period is often shorter than the global surgical period (e.g., 60 days), which creates a time period gap between when the reimbursement can be paid and when new procedure billing data can be submitted. This can create data discrepancies in the billing data because the urgency rule may only apply to paying the reimbursement once the bill is received, without an urgency rule to make sure the data is complete in the system.

As an illustrative example, a patient may undergo a surgery with a healthcare provider (e.g., first procedure) and return to the healthcare provider for dressings changes or other office visit (e.g., second procedure). The healthcare provider may accidentally or fraudulently submit a bill to an insurance computer system for the second procedure without submitting a bill for the first procedure. The bill for the second procedure may be paid by the insurance provider to the healthcare provider, in order to meet various time urgency rules for providing a reimbursement for medical bills to healthcare providers (e.g., to comply with the expedited reimbursement period, etc.). After the bill is paid for the second procedure, the healthcare provider may submit a bill corresponding with the first procedure. This bill corresponding with the first procedure may be submitted within the global surgical period, but after the second procedure has been paid. The second procedure may not be associated or linked to the first procedure in a standard insurance computer system, since the bill for the second procedure was submitted prior to the insurance computer system receiving any information about the first procedure and thus having no originating data entry prior in time to subsequently received bill data for the first procedure.

Additionally, a standard insurance computer system may automatically reimburse the second procedure because an identifier associated with the second procedure matches a list of covered procedures (e.g., payable by the insurance computer system to the healthcare provider). However, the rules for reimbursing the second procedure may be different when it is viewed with or without the first procedure. For example, the cost of an office visit may be one hundred dollars, but the cost may be reduced to zero when the office visit is in conjunction with a surgery. In either of these examples, the quality of the data is key in determining the automated actions of the insurance computer system for determining whether to issue reimbursements for procedures as well as the correct reimbursement amount.

In some embodiments, an improved insurance computer system may enhance the data and provide more accurate reimbursements for medical bills to healthcare providers. For example, after the second procedure is paid and the bill for the related first procedure is received, the system may automatically search through the data records to find related records even after one or more have been paid (e.g., run in real time from receiving the bill from the healthcare provider, etc.). The first procedure and the second procedure may be correlated based on a matching service code. Once the two procedures are correlated in the system, the improved insurance computer system may initiate an action associated with the identified improper reimbursement. The action may, for example, issue an explanation of benefits (EOB) or explanation of reimbursement (EOR) that identifies the improper payment and/or a request to return the improperly dispersed reimbursement from the healthcare provider. Another action may, for example, issue an invoice to the healthcare provider to return payment to the improved insurance computer system. Another action may, for example, subtract funds from future payments (e.g., ten thousand total for the surgical procedure, then subtract one thousand from the total so that nine thousand is left to correlate to the surgical procedure for secondary, reimbursable procedures).

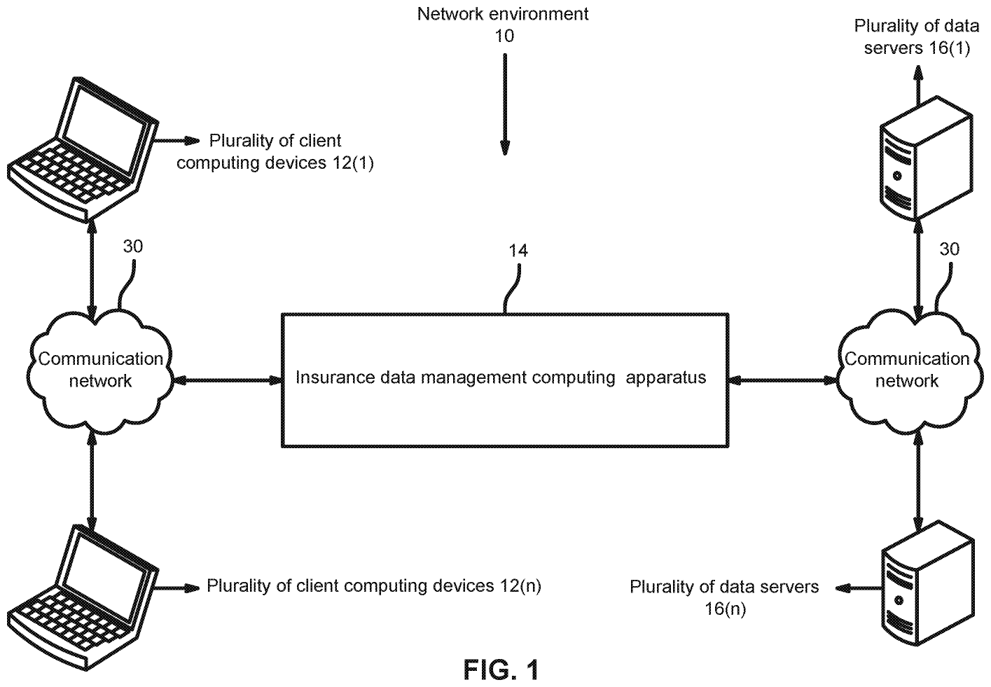

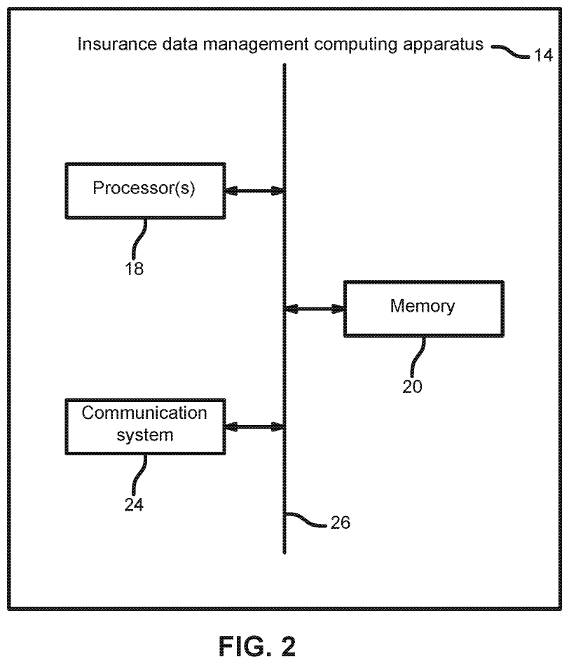

An environment 10 with an example of an insurance data management computing apparatus 14 is illustrated in FIGS. 1-2. In this particular example, the environment 10 includes the insurance data management computing apparatus 14, client computing devices 12(l)-12(n), plurality of data servers 16(l)-16(n) coupled via one or more communication networks 18, although the environment could include other types and numbers of systems, devices, components, and/or other elements as is generally known in the art and will not be illustrated or described herein. This technology provides a number of advantages including providing methods, non-transitory computer readable medium, and apparatuses that identify and prevent incorrect and/or potentially fraudulent medical data. By way of example, a potentially fraudulent medical data can include bills that are submitted for the follow-up visits post surgery, prior to submitting the actual bill data of the surgery.

Referring more specifically to FIGS. 1-2, the insurance data management computing apparatus 14 is programmed to provide efficient methods to identify and prevent incorrect and/or potentially fraudulent medical data, although the apparatus can perform other types and/or numbers of functions or other operations and this technology can be utilized with other types of claims. In this particular example, the insurance data management computing apparatus 14 includes a processor 18, a memory 20, and a communication system 24 which are coupled together by a bus 26, although the insurance data management computing apparatus 14 may comprise other types and/or numbers of physical and/or virtual systems, devices, components, and/or other elements in other configurations.

The processor 18 in the insurance data management computing apparatus 14 may execute one or more programmed instructions stored in the memory 20 for identifying and preventing incorrect and/or potentially fraudulent medical data as illustrated and described in the examples herein, although other types and numbers of functions and/or other operations can be performed. The processor 18 in the insurance data management computing apparatus 14 may include one or more central processing units and/or general purpose processors with one or more processing cores, for example.

The memory 20 in the insurance data management computing apparatus 14 stores the programmed instructions and other data for one or more aspects of the present technology as described and illustrated herein, although some or all of the programmed instructions could be stored and executed elsewhere. A variety of different types of memory storage devices, such as a random access memory (RAM) or a read only memory (ROM) in the system or a floppy disk, hard disk, CD ROM, DVD ROM, or other computer readable medium which is read from and written to by a magnetic, optical, or other reading and writing system that is coupled to the processor 18, can be used for the memory 20.

The communication system 24 in the insurance data management computing apparatus 14 operatively couples and communicates between one or more of the client computing devices 12(l)-12(n) and one or more of the plurality of data servers 16(l)-16(n), which are all coupled together by one or more of the communication networks 30, although other types and numbers of communication networks or systems with other types and numbers of connections and configurations to other devices and elements can be used. By way of example only, the communication networks 30 can use TCP/IP over Ethernet and industry-standard protocols, including NFS, CPFS, SOAP, XML, LDAP, SCSI, and SNMP, although other types and numbers of communication networks, can be used. The communication networks 30 in this example may employ any suitable interface mechanisms and network communication technologies, including, for example, any local area network, any wide area network (e.g., Internet), teletraffic in any suitable form (e.g., voice, modem, and the like), Public Switched Telephone Network (PSTNs), Ethernet-based Packet Data Networks (PDNs), and any combinations thereof and the like.

In this particular example, each of the client computing devices 12(l)-12(n) may submit requests for medical claims or bills to the insurance data management computing apparatus 14, although the requests can be obtained by the insurance data management computing apparatus 14 in other manners and/or from other sources. Each of the client computing devices 12(l)-12(n) may include a processor, a memory, user input device, such as a keyboard, mouse, and/or interactive display screen by way of example only, a display device, and a communication interface, which are coupled together by a bus or other link, although each may have other types and/or numbers of other systems, devices, components, and/or other elements.

The plurality of data servers 16(l)-16(n) may store and provide data associated with different insurance carriers, by way of example only, to the insurance data management computing apparatus 14 via one or more of the communication networks 30, for example, although other types and/or numbers of storage media in other configurations could be used. In this particular example, each of the plurality of data servers 16(l)-16(n) may comprise various combinations and types of storage hardware and/or software and represent a system with multiple network server devices in a data storage pool, which may include internal or external networks. Various network processing applications, such as CIFS applications, NFS applications, HTTP Web Network server device applications, and/or FTP applications, may be operating on the plurality of data servers 16(l)-16(n) and may transmit data in response to requests from the insurance data management computing apparatus 14. Each the plurality of data servers 16(l)-16(n) may include a processor, a memory, and a communication interface, which are coupled together by a bus or other link, although each may have other types and/or numbers of other systems, devices, components, and/or other elements.

Although the exemplary network environment 10 with the insurance data management computing apparatus 14, the agent computing devices 12(l)-12(n), the plurality of data servers 16(l)-16(n), and the communication networks 30 are described and illustrated herein, other types and numbers of systems, devices, components, and/or elements in other topologies can be used. It is to be understood that the systems of the examples described herein are for exemplary purposes, as many variations of the specific hardware and software used to implement the examples are possible, as will be appreciated by those skilled in the relevant art(s).

In addition, two or more computing systems or devices can be substituted for any one of the systems or devices in any example. Accordingly, principles and advantages of distributed processing, such as redundancy and replication also can be implemented, as desired, to increase the robustness and performance of the devices, apparatuses, and systems of the examples. The examples may also be implemented on computer system(s) that extend across any suitable network using any suitable interface mechanisms and traffic technologies, including by way of example only teletraffic in any suitable form (e.g., voice and modem), wireless traffic media, wireless traffic networks, cellular traffic networks, G3 traffic networks, Public Switched Telephone Network (PSTNs), Packet Data Networks (PDNs), the Internet, intranets, and combinations thereof.

The examples also may be embodied as a non-transitory computer readable medium having instructions stored thereon for one or more aspects of the present technology as described and illustrated by way of the examples herein, as described herein, which when executed by the processor, cause the processor to carry out the steps necessary to implement the methods of this technology as described and illustrated with the examples herein.

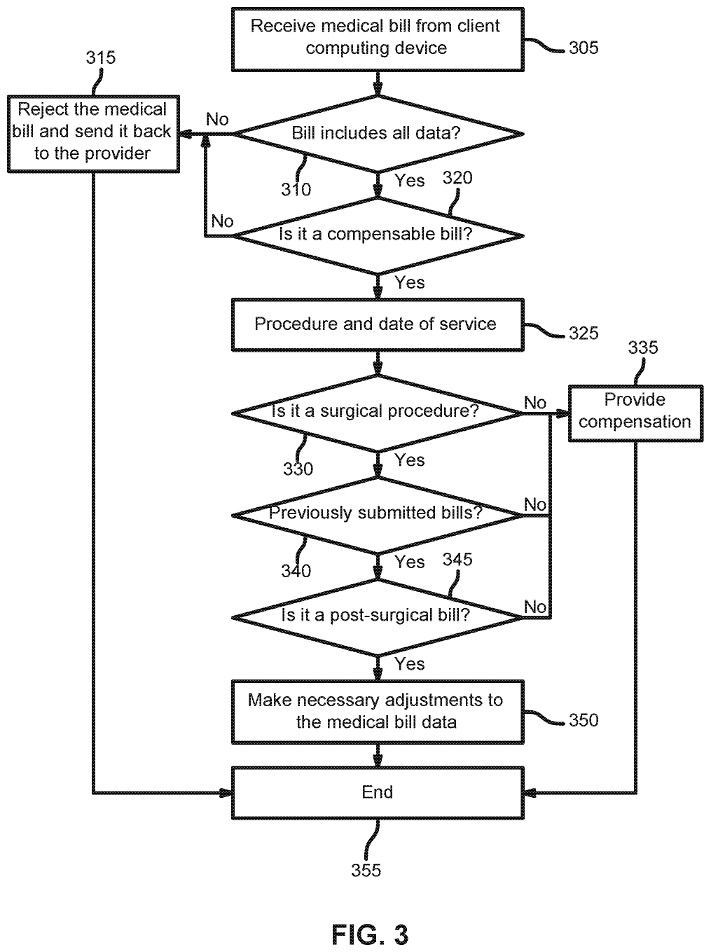

An example of a method for identifying and preventing incorrect and/or potentially fraudulent medical data will now be described with reference to FIGS. 1-4B. In particular, referring to FIG. 3 the exemplary method begins at step 305 where the insurance data management computing apparatus 14 receives a medical bill data or claim from one of the plurality of client computing devices 1c2(l)-12(n), although the insurance data management computing apparatus 14 can receive other types or requests from other devices. In this example, the medical bill data includes data associated with a service that was provided to the patient such as date of service, service code, service description, and money charged, although the medical bill data can include other types or amounts of data.

Next in step 310, the insurance data management computing apparatus 14 determines if the received medical bill data includes all the required data for further processing. By way of example, the insurance data management computing apparatus 14 can compare the received medical bill data against a standard medical bill data to determine if the received medical bill data includes all the data, although the insurance data management computing apparatus 14 can determine use other techniques. Accordingly, if the insurance data management computing apparatus 14 determines that the received medical bill data does not include all the data, then the No branch is taken to step 315. In step 315, the insurance data management computing apparatus 14 rejects the received bill data and sends the received medical bill data with optional related commentary data identifying the issue or issues back to the requesting one of the plurality of client computing devices 12(l)-12(n).

However, if back in step 310 the insurance data management computing apparatus 14 determines that the bill data includes all the required data for further processing, then the Yes branch is taken to step 320. In step 320, the insurance data management computing apparatus 14 determines if the received bill data is a compensable bill data by identifying and correlating contents of the received bill data against a medical insurance database. If the service code in the received bill data matches with the service code in the medical insurance database, then the insurance data management computing apparatus 14 determines that the received bill data is compensable. However, if the service code in the received medical bill data is not present in the medical insurance database, then the insurance data management computing apparatus 14 determines that the received bill data is not compensable. Alternatively, other techniques can be used to determine if the received bill data is a compensable bill.

Accordingly, if the insurance data management computing apparatus 14 determines that the bill data is not compensable, then a No branch is taken to step 315 where the received medical bill data is rejected. However, if in step 320 the insurance data management computing apparatus 14 determines that the received bill data is a compensable bill, then the Yes branch is taken to step 325.

In step 325, the insurance data management computing apparatus 14 identifies the data associated with the service that was provided to the patient and the date of the service from the received medical bill data, although the insurance data management computing apparatus 14 can identify other types and/or amounts of information from the received medical bill data. In this example, the service that was provided can be identified based on a service code in the received medical bill data and obtain additional information associated with the service code from the medical insurance database, although the additional information can be obtained from other locations.

In step 330, the insurance data management computing apparatus 14 determines if the submitted medical bill data is associated with a surgical procedure that was performed on a patient based on the data identified in step 325, although the insurance data management computing apparatus 14 can use other techniques to make the determination. Accordingly, if the insurance data management computing apparatus 14 determines that the received medical bill data is not relating to the surgical procedure, then the No branch is taken to step 335. In step 335, the insurance data management computing apparatus 14 adds textual data to the received medical bill data to provide compensation.

However, if in step 330 the insurance data management computing apparatus 14 determines that the received medical bill data is relating to the surgical procedure, then the Yes branch is taken to step 340. In step 340, the insurance data management computing apparatus 14 determines if there is one or more previously submitted medical bills relating to the received medical bill data.

In this example, the insurance data management computing apparatus 14 may determine the one or more previously submitted medical bills from the date of service in the received medical bill data and submitted prior to the received medical bill data, although the insurance data management computing apparatus 14 can use other parameters to determine the one or more previously submitted medical bills. Additionally, the insurance data management computing apparatus 14 can use data such as the service code, the service provider code, service provider data, or service description from the received medical bill data to determine if the one or more previously submitted medical bills relates to the received medical bill data. Accordingly, if the insurance data management computing apparatus 14 determines that there are no previously submitted bills, then the No branch is taken to step 335 that is illustrated above.

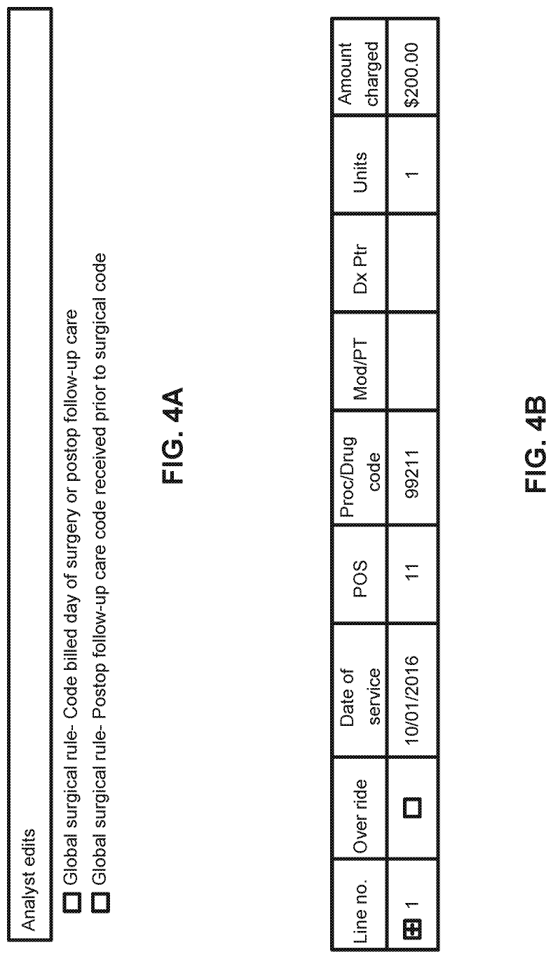

However, if back in step 340, the insurance data management computing apparatus 14 determines that there is one or more previously submitted bills relating to the received medical bill data, then the Yes branch is taken to step 345. In step 345, the insurance data management computing apparatus 14 determines if the determined one or more previously submitted bills are post-surgical bills using the techniques illustrated in steps 330 and 340, although the insurance data management computing apparatus 14 can use other techniques to make the determination. By way of example, FIG. 4A illustrates a rule to determine for the post-surgical bills. Accordingly, if the insurance data management computing apparatus 14 determines that the determined one or more previously submitted bills are not post-surgical bills, then the No branch is taken to step 335.

However, if the insurance data management computing apparatus 14 determines that the determined one or more previously submitted bills are post-surgical bills, then the Yes branch is taken to step 350. In step 350, the insurance data management computing apparatus 14 can make necessary adjustments to the received medical bill data by subtracting the total amount that was previously paid in the determined one or more previously submitted bills, from the received medical bill data. By way of example, FIG. 4B illustrates an example of one such adjustment. Alternatively in other examples, the insurance data management computing apparatus 14 can perform other types of adjustments to the received medical bill data and the exemplary method ends at step 355. By making the necessary adjustments to the received medical bill data, the disclosed technology is able to identify incorrect and potentially fraudulent bill data submitted and prevents compensation being paid for the incorrect and/or potentially fraudulent bills.

Having thus described the basic concept of the invention, it will be rather apparent to those skilled in the art that the foregoing detailed disclosure is intended to 10 be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

What is claimed is:

1. A method for more accurately managing processing of medical bill data, the method comprising:
identifying, by the insurance data management computing apparatus, previously submitted bill data associated with received medical bill data from a client based on one or more service data parameters in the received medical bill data and the identified previously submitted bill data;
determining, by the insurance data management computing apparatus, when the received medical bill data is erroneous medical bill data based on the identified previously submitted bill data and a time period between the previously submitted bill data and the received medical bill data;
classifying, by the insurance data management computing apparatus, the received medical bill data as a follow-up procedure bill data when the received medical bill data is determined to be an erroneous medical bill data; and
restricting, by the insurance data management computing apparatus, compensation data for the received medical bill data classified as the follow-up procedure bill data.

2. The method as set forth in claim 1 wherein the received medical bill data is determined to be the erroneous medical bill data when a service date of received medical bill data is within the time period of the identified previously submitted bill data.

3. The method as set forth in claim 1 further comprising determining, by the insurance data management computing apparatus, when the received medical bill data is associated with a surgical procedure prior to determining when the medical bill data is the erroneous medical bill data.

4. The method as set forth in claim 1 further comprising, determining, by the insurance data management computing apparatus, when the received medical bill data is a compensable medical bill data.

5. The method as set forth in claim 1 further comprising identifying, by the insurance data management computing apparatus, a service code, the service date, a service description and service provider data from the received medical bill data and the identified one or more previously submitted bill data.

6. The method as set forth in claim 1 wherein the service data comprises the service code, the service date, the service description and service provider data.

7. A non-transitory computer readable medium having stored thereon instructions for more accurately managing processing of medical bill data comprising executable code, which when executed by at least one processor, cause the processor to:
identify previously submitted bill data associated with received medical bill data from a client based on one or more service data parameters in the received medical bill data and the identified previously submitted bill data;
determine when the received medical bill data is erroneous medical bill data based on the identified previously submitted bill data and a time period between the previously submitted bill data and the received medical bill data;
classify the received medical bill data as a follow-up procedure bill data when the received medical bill data is determined to be an erroneous medical bill data; and
restrict compensation data for the received medical bill data classified as the follow-up procedure bill data.

8. The medium as set forth in claim 7 wherein the received medical bill data is determined to be the erroneous medical bill data when a service date of received medical bill data is within the time period of the identified previously submitted bill data.

9. The medium as set forth in claim 7 further comprising determine when the received medical bill data is associated with a surgical procedure prior to determining when the medical bill data is the erroneous medical bill data.

10. The medium as set forth in claim 7 further comprises determine when the received medical bill data is a compensable medical bill data.

11. The medium as set forth in claim 7 further comprising, identifying a service code, the service date, a service description and service provider data from the received medical bill data and the identified one or more previously submitted bill data.

12. The medium as set forth in claim 7 wherein the service data comprises the service code, the service date, the service description and service provider data.

13. An insurance data management computing apparatus comprising:
a processor; and
a memory coupled to the processor which is configured to be capable of executing programmed instructions comprising and stored in the memory to:
identify previously submitted bill data associated with received medical bill data from a client based on one or more service data parameters in the received medical bill data and the identified previously submitted bill data;
determine when the received medical bill data is erroneous medical bill data based on the identified previously submitted bill data and a time period between the previously submitted bill data and the received medical bill data;
classify the received medical bill data as a follow-up procedure bill data when the received medical bill data is determined to be an erroneous medical bill data; and
restrict compensation data for the received medical bill data classified as the follow-up procedure bill data.

14. The apparatus as set forth in claim 13 wherein the received medical bill data is determined to be the erroneous medical bill data when a service date of received medical bill data is within the time period of the identified previously submitted bill data.

15. The apparatus as set forth in claim 13 wherein the processor is further configured to be capable of executing the stored programmed instructions to determine when the received medical bill data is associated with a surgical procedure prior to determining when the medical bill data is the erroneous medical bill data.

16. The apparatus as set forth in claim 13 wherein the processor is further configured to be capable of executing the stored programmed instructions to determine when the received medical bill data is a compensable medical bill data.

17. The apparatus as set forth in claim 13 wherein the processor is further configured to be capable of executing the stored programmed instructions to identifying a service code, the service date, a service description and service provider data from the received medical bill data and the identified one or more previously submitted bill data.

18. The apparatus as set forth in claim 13 wherein the service data comprises the service code, the service date, the service description and service provider data.
